(19) United States
(12) Patent Application Publication
(10) Pub. No.: US 2015/0258317 A1
(43) Pub. Date: Sep. 17, 2015

(54) Hydrotherapy Covering

(71) Applicants: Kathleen Kanuk, Chardon, OH (US); Daniel Kanuk, Chardon, OH (US)

(72) Inventors: Kathleen Kanuk, Chardon, OH (US); Daniel Kanuk, Chardon, OH (US)

(21) Appl. No.: 14/658,878

(22) Filed: Mar. 16, 2015

Related U.S. Application Data
(60) Provisional application No. 61/954,021, filed on Mar. 17, 2014.

(51) Int. Cl. A61M 35/00

(52) U.S. Cl. CPC A61M 35/003

(57) ABSTRACT

An antiseptic hydrotherapy covering for hands and feet is provided. The covering includes an interior layer and an exterior layer. The covering defines a reservoir having an open end to receive a fluid and an extremity of a user. The interior layer includes a plurality of evenly spaced air-filled hemispheres. The reservoir has a volume surrounded by the plurality of evenly spaced air-filled hemispheres. A fastener is disposed on the open end of the reservoir of the covering. The fastener is secured around the open end to enclose a fluid and an extremity within the covering. The covering provides an effective insulator allowing the fluid contained within the covering to maintain its temperature for a longer period of time as compared to traditional soaking methods.

### CROSS REFERENCE TO RELATED APPLICATION

This application claims the benefit of U.S. Provisional Application No. 61/954,021 filed on Mar. 17, 2014. The above identified patent application is herein incorporated by reference in its entirety to provide continuity of disclosure.

### FIELD OF THE INVENTION

The invention generally relates to a hydrotherapy covering. More particularly, the present invention relates to an antiseptic hydrotherapy covering including a covering adapted to receive the extremities of a user as well as a fluid within the interior volume of the covering. The interior layer of the covering comprises a plurality of evenly spaced air-filled hemispheres that protrude from the interior layer that serve as an effective insulator allowing the fluid contained within the covering to maintain its temperature.

### BACKGROUND OF THE INVENTION

Various aliments afflict the feet and hands that are painful and uncomfortable. Many of them, such as callouses, corns, rashes, thick toe nails, scaly skin, athlete's foot and dry skin may be successfully treated by the application of therapeutic lotions, creams, mud packs, poultices, and like compositions. Hydrotherapy is also effective in stimulating the inherent healing powers of the body. Traditionally, however, these treatments have required the patient to devote periods of time exclusively to receiving treatment. Often they have required the person receiving treatment to travel to a particular location or establishment where treatment is provided, at times with the assistance and associated cost of an attendant or therapist.

Certain injuries are ideally treated by being soaking in water or saline solution for an extended period of time. Hydrotherapy can be an effective means of reducing swelling, cleaning open wounds, and increasing blood flow to affected areas. Although highly medically beneficial, hydrotherapy can be a taxing process because individuals are generally forced to soak their extremities in pans or basins. Using basins full of water or saline solution can be very messy because it is so easy to knock the basin over or otherwise spill the water. Furthermore, it can be quite uncomfortable for an individual to keep his or her wounded extremity submerged within the basin for an extended period of time because the individual may have to contort his or her body to soak the extremity. Finally, water kept within an open basin quickly equilibrates to room temperature, thereby reducing the effectiveness of both warm and cold soaks. Therefore, there is a need in the prior art for a device that gives individuals the ability to soak their wounded extremities for an extended period of time, in a temperature-controlled and tidy manner.

Devices have been disclosed in the prior art that relate to hand and feet treatment systems. These include devices that have been patented and published in patent application publications. U.S. Pat. No. 4,622,035, U.S. Pat. No. 4,775,372, U.S. Pat. No. 7,938,811, U.S. Pat. No. 6,792,701, and U.S. Pat. No. 6,896,667 disclose customized shoes, gloves, foot and hand pads, sponge rubber stick-on pads for corns and callouses and similar items adapted to make standing or walking more comfortable.

These devices, however, fail to disclose a hydrotherapeutic covering comprising a covering having an interior layer and an exterior layer, wherein the interior layer comprises a plurality of evenly spaced air-filled hemispheres that protrude from the interior layer. Additionally, these devices fail to disclose that the evenly spaced air-filled hemispheres protrude inwardly from the interior layer to contact a user's skin.

In light of the devices disclosed in the prior art, it is submitted that the present invention substantially diverges in design elements from the prior art and consequently it is clear that there is a need in the art for an improvement to existing hydrotherapeutic coverings. In this regard the instant invention substantially fulfills these needs.

### SUMMARY OF THE INVENTION

In view of the foregoing disadvantages inherent in the known types of hydrotherapy treatments now present in the prior art, the present invention provides a hydrotherapy covering wherein the same can be utilized for providing convenience for the user when treating ailments of the hands and feet by soaking extremities in water.

It is therefore an object of the present invention to provide a new and improved antiseptic hydrotherapy covering that has all of the advantages of the prior art and none of the disadvantages.

It is another object of the present invention to provide an antiseptic hydrotherapy covering for hands and feet including a covering having an interior layer and an exterior layer. The interior layer comprises a plurality of evenly spaced air-filled hemispheres that protrude from the interior layer.

It is yet another object of the present invention to provide an antiseptic hydrotherapy covering including a reservoir having an interior volume surrounded by the plurality of evenly spaced air-filled hemispheres. The reservoir has a closed end and an open end. The interior volume of the reservoir is adapted to receive an extremity of a user via the open end. The plurality of evenly spaced air-filled hemispheres provides an insulating layer for contents within the interior volume of the reservoir.

It is still yet another object of the present invention to provide an antiseptic hydrotherapy covering including an adjustable fastener disposed on the open end of the reservoir. The fastener is adapted to enclose the extremity within the interior volume as well as other contents placed within the bag, such as a fluid. The fastener may be a hook and loop, an adjustable strap, or adhesive.

A further object of the present invention is to provide an antiseptic hydrotherapy covering wherein the plurality of evenly spaced air-filled hemispheres of the interior layer protrude inwardly into the reservoir. The air-filled hemispheres of the interior layer lay flush against the skin of the user.

Another object of the present invention is to provide an antiseptic hydrotherapy covering wherein the covering has an interior volume corresponding to a boot or a glove removably attachable to the feet or hands of a user, respectively.

Yet another object of the present invention is to provide an antiseptic hydrotherapy covering including a release valve. The release valve allows a fluid contained within the reservoir of the covering to be drained when treatment is terminated.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the covering in a glove configuration according to one embodiment of the present invention.

FIG. 2 shows a perspective view of the covering in a boot configuration according to one embodiment of the present invention.

FIG. 3 shows an overhead cross-sectional view of the covering according to one embodiment of the present invention.

FIG. 4 shows a close-up view of the fastener of the covering according to one embodiment of the present invention.

### DETAILED DESCRIPTION OF THE INVENTION

Reference is made herein to the attached drawings. Like reference numerals are used throughout the drawings to depict like or similar elements of the hydrotherapy covering. For the purposes of presenting a brief and clear description of the present invention, the preferred embodiment will be discussed as used for treating ailments and wounds on the hands and feet. The figures are intended for representative purposes only and should not be considered to be limiting in any respect.

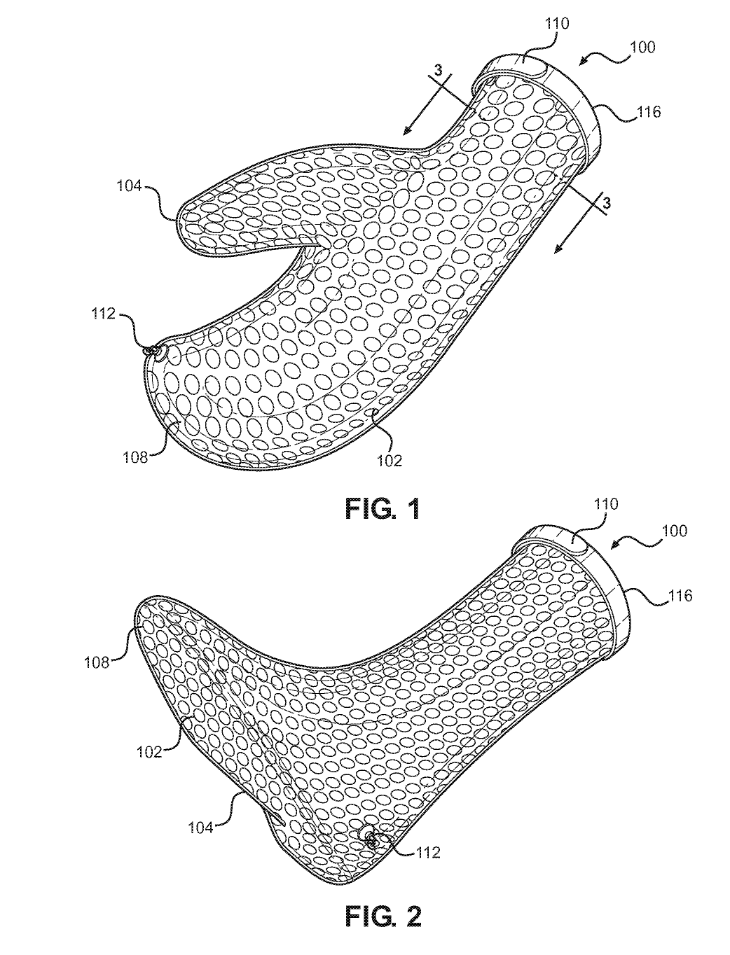

Referring now to FIGS. 1 and 2, there are shown perspective views of the covering in a glove and boot configuration, respectively. The antiseptic hydrotherapy covering includes a covering 100 having an interior layer 102, an exterior layer 104, and a reservoir within the covering 100. The covering 100 is adapted to snugly fit over an individual's hands or feet within the reservoir. The interior layer 102 comprises a plurality of evenly spaced air-filled hemispheres, similar to bubble wrap, that protrude from the interior layer 102. The interior layer 102 provides a cushioning surface that lightly rests against the user's wounded extremity. The interior layer 102 serves as an effective insulator, allowing the fluid contained within the covering 100 to maintain its temperature for a longer period of time as compared to traditional soaking methods. The covering 100 is adapted for a single use, wherein waste water can be removed. In one embodiment, the covering 100 is in a glove-like configuration that has a portion for a user's fingers and a portion for the thumb. In another embodiment, the covering 100 is in a boot-like configuration that has a portion for a user's toes and a portion for the heel.

The exterior layer 104 of the covering 100 comprises a waterproof liner. The waterproof liner provides additional insulation for the covering 100 and prevents water from escaping the interior volume of the reservoir. The exterior layer 104 may also include a release valve 112. The release valve 112 extends through the exterior layer 104 and the interior layer 102 to form an aperture in the reservoir. The release valve 112 provides an aperture to drain fluid from the reservoir after a treatment cycle. The release valve 112 is selectively opened and closed by removing a rubber stopper coupled to the release valve 112. It is contemplated that the covering 100 may be provided without an exterior layer 104. In this configuration, the interior layer is in direct contact with the atmosphere. It is also contemplated that an insulation layer may be provided between the exterior layer 104 and the interior layer 102.

The covering 100 includes a reservoir having a closed end 108 and an open end 116 configured to receive the extremities of a user. In use, individuals fill the reservoir with either warm or cold water, as desired, and then secure the covering 100 to their hands or feet. The reservoir provides a sufficient volume to soak the user's hand and feet in the warm or cold water. A fastener 110 is disposed on the open end 116 of the reservoir of the covering 100. The fastener 110 is tightened around the open end 116 of reservoir and provides a water tight seal preventing any water from leaking out of the covering 100. The fastener 110 can be an elongated strap adapted to encircle a user's ankle or wrist, depending on the embodiment.

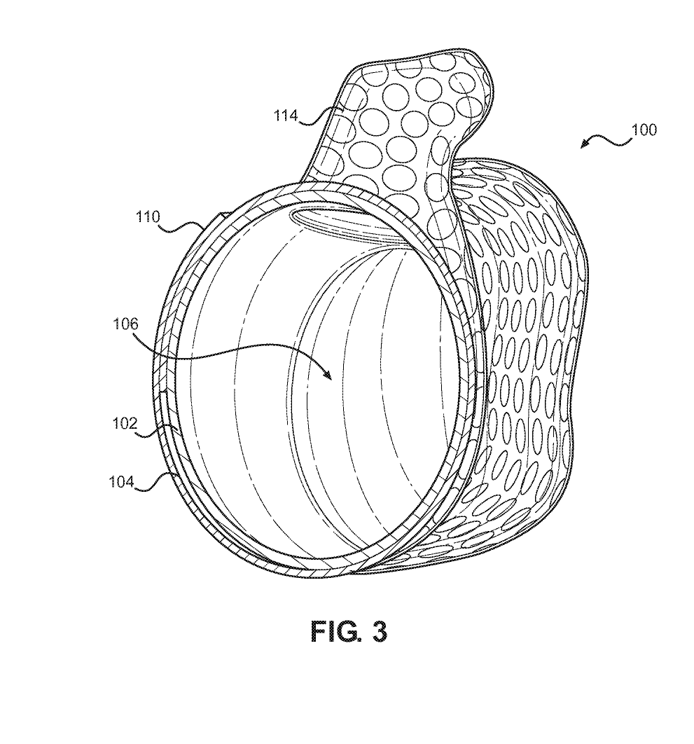

Referring now to FIG. 3, there is shown an overhead cross-sectional view of the covering according to one embodiment of the present invention. The covering 100 comprises a plurality of layers that define a hollow interior volume of the reservoir 106. The interior volume is configured to receive an extremity as well as a volume of liquid, such as a saline solution or warm water. The covering 100 includes at least an exterior layer 104, and interior layer 102, and a fastener 110 that surround the hollow interior volume.

The interior layer 102 comprises a plurality of evenly spaced air-filled hemispheres 114. The evenly spaced air-filled hemispheres 114 can be arranged to surround the entire periphery of the covering 100. The interior layer 102 defines the contour of the interior volume of the reservoir 106. The evenly spaced air-filled hemispheres 114 of the interior layer protrude inwardly into the reservoir 106. The air-filled hemispheres 114 provide a cushioning surface that lightly rests against the user's wounded extremity. It also contemplated that the air-filled hemispheres 114 of the interior layer 102 protrude outwardly providing a dual sided covering 100 with evenly spaced air-filled hemispheres that protrude in both directions from the interior layer 102.

The reservoir 106 has an interior volume surrounded by the plurality of evenly spaced air-filled hemispheres 114. The reservoir 106 has a closed end and an open end. The closed end receives the terminating end of the extremity. The open end is adapted to receive an extremity of a user in the interior volume of the reservoir 106. In use, a user first places his or her hands or feet into the covering 100 via the open end. Next, the reservoir 106 is filed with a fluid, such as saline solution or warm water, preferably to a designated fill line. The fastener 110 is then secured around the user's extremity to enclose both the extremity and the fluid within the covering 100.

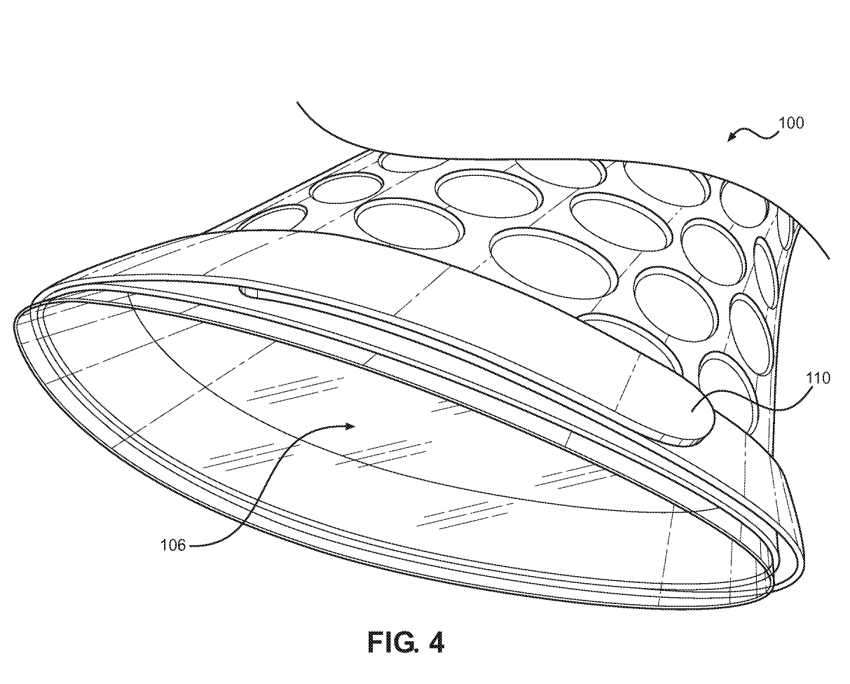

Referring now to FIG. 4, there is shown a close-up view of the fastener of the covering according to one embodiment of the present invention. The fastener 110 is disposed on the open end of the reservoir 106. The fastener is adapted to enclose the extremity within the interior volume and provide a water tight seal. The fastener 110 is pulled around an extremity to urge the open end of the reservoir 106 against the skin of a user. The open end rests flush against the user's skin, enclosing the extremity within the covering 100. The fastener 110 may be a hook and loop fastener, an adjustable strap, or an adhesive. In one embodiment, the fastener 110 is an elongated strap that includes a hook and loop fastening material thereon so that the strap can be fitted to users of different ankle and wrist sizes.

In one embodiment, the antiseptic hydrotherapy covering for hands and feet comprises a covering 100 including a single layer having a plurality of evenly spaced air-filled hemispheres. The plurality of evenly spaced air-filled hemispheres of the single layer protrude outwardly away from a user's skin. The single layer defines a reservoir having an interior volume. The reservoir has a closed end and an open end. The interior volume of the reservoir is adapted to receive an extremity of a user via the open end. A fastener disposed on the open end of the reservoir is configured to close the reservoir against the user's skin.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An antiseptic hydrotherapy covering for hands and feet, comprising:
a covering defining a reservoir having an open end, wherein the covering includes an interior layer and an exterior layer, wherein the interior layer comprises a plurality of evenly spaced air-filled hemispheres;
the reservoir adapted to receive an extremity of a user via the open end;
a fastener disposed on the open end of the covering, wherein the fastener is adapted to enclose the extremity within the interior volume;
wherein the plurality of evenly spaced air-filled hemispheres of the interior layer protrude inwardly into the reservoir.

2. The hydrotherapy covering of claim 1, wherein the covering further comprises a release valve adapted to allow fluid stored within said reservoir to drain therefrom.

3. The hydrotherapy covering of claim 1, wherein the fastener is an elongated strap having a hook and loop fastener thereon configured to encircle a user's limb.

4. The hydrotherapy covering of claim 1, wherein the covering resembles a glove shape.

5. The hydrotherapy covering of claim 1, wherein the covering resembles a boot shape.

6. The hydrotherapy covering of claim 1, wherein the fastener is a strap including an adhesive thereon.

7. The hydrotherapy covering of claim 1, wherein the exterior layer comprises a waterproof liner.

8. An antiseptic hydrotherapy covering for hands and feet, comprising:
a covering including a single layer comprising a plurality of evenly spaced air-filled hemispheres;
a reservoir having an interior volume is defined by the single layer, the reservoir having a closed end and an open end, wherein the interior volume of the reservoir is adapted to receive an extremity of a user via the open end;
a fastener disposed on the open end of the reservoir;
wherein the plurality of evenly spaced air-filled hemispheres of the single layer protrude outwardly.

9. The hydrotherapy covering of claim 8, wherein the single layer includes a release valve.

10. The hydrotherapy covering of claim 8, wherein the covering has a glove shape.

11. The hydrotherapy covering of claim 8, wherein the covering has a boot shape.
